(19) United States
(12) Patent Application Publication
(10) Pub. No.: US 2018/0280059 A1
(43) Pub. Date: Oct. 4, 2018

(54) PRP Delivery Device for Vaginal Tightening

(71) Applicant: Eclipse Aesthetics, LLC, The Colony, TX (US)

(72) Inventor: Thomas Michael O'Brien, III, Dallas, TX (US)

(21) Appl. No.: 15/782,849

(22) Filed: Oct. 13, 2017

Related U.S. Application Data
(60) Provisional application No. 62/408,324, filed on Oct. 14, 2016.

(51) Int. Cl. A61B 17/42; A61K 35/16; A61M 25/10; A61M 37/00

(52) U.S. Cl. CPC A61B 17/42; A61K 35/16; A61K 35/19; A61M 37/0015; A61M 25/10185

(57) ABSTRACT

Disclosed is a system and related method for delivering platelet-rich plasma to the vaginal cavity to promote vaginal rejuvenation. Such a system may comprise an outer balloon comprising a plurality of recessed microneedles, an inner balloon inside the outer balloon, a PRP pump, connected to a space between the outer balloon and the inner balloon and connected to a PRP source, and a fluid pump, connected to an opening of the inner balloon. The PRP pump of the system may inject PRP from the PRP source into the space between the outer balloon and the inner balloon, and then the fluid pump may inflate the inner balloon to force the PRP inside the space between the outer balloon and the inner balloon through the plurality of recessed microneedles into the vaginal cavity.

### CROSS-REFERENCE TO RELATED APPLICATION

This application claims the benefit of and priority to U.S. Provisional Patent Application No. 62/408,324 filed Oct. 14, 2016, the technical disclosure of which is hereby incorporated herein by reference.

### BACKGROUND OF THE INVENTION

### Technical Field

The present invention relates to an apparatus for delivery of platelet-rich plasma (PRP) to a body cavity, and more specifically to the vagina and vaginal areas for the purpose of tightening vaginal tissues in the vaginal area, and a method for delivery of PRP to the vaginal areas.

### Description of Related Art

Platelet-rich plasma is a blood plasma that has been enriched with platelets, and contains different components that triggers healing responses in human tissue. PRP is used extensively in dermatology, orthopedics, dentistry, and other medical fields. The theory behind PRP therapy is that certain growth factors are present in healing various injuries, and by replicating these growth factors in certain concentrations in PRP therapy injections, doctors can accelerate a patient's healing process, which can, among other things, tighten and rejuvenate tissue, or stimulate growth of new cells at the injection site. When specifically applied to the vaginal area of female patients, PRP therapy may improve urinary incontinence and sexual response by tightening the tissues in the treated area.

Currently, PRP therapy involves extracting platelets from a patient's own blood via centrifugation and reinjecting the platelets into target areas. Growth factors may also be harvested from the separated blood components, and these growth factors include platelet derived growth factor, vascular endothelial growth factor, transforming growth factor betal, keratinocyte growth factor, fibroblast growth factor, and epidermal growth factor. Once platelets and growth factors are separated from the other blood components, a doctor injects the combination of platelets and growth factors into the target area.

The current state of the art of PRP therapy for use to the vaginal areas of female patients is the 0-shot. This procedure involves injecting the PRP into the vagina using hypodermic syringes after application of extensive pain-numbing on and inside the vagina. Another method of PRP therapy for use to the vaginal areas of female patients is PRP Vaginal Rejuvenation by Defy Medical. With PRP Vaginal Rejuvenation, pain numbing is applied on and inside the vagina before a solution is injected into the fat pad of the supra pubic region, so that a doctor has easier access to the vaginal areas. The PRP is then injected via hypodermic syringes into the clitoral and urethral area, and is optionally injected into the G-spot (Grafenberg spot) of the vagina.

The current state of the art for PRP therapy for use to vaginal areas is limited to PRP injections with hypodermic syringes to limited treatment areas after extensive application of pain numbing medication. What is needed is a system, device, or method that treats a larger area and requires application of less pain numbing medication.

### BRIEF SUMMARY

To overcome the deficiencies in the prior art, the disclosed principles provide for various embodiments of a PRP delivery system for vaginal rejuvenation.

In accordance with a first embodiment, a PRP delivery system for vaginal rejuvenation is provided. The PRP delivery system for vaginal rejuvenation may comprise an outer balloon comprising a plurality of recessed microneedles, an inner balloon inside the outer balloon, a PRP pump, connected to a space between the outer balloon and the inner balloon and connected to a PRP source, and a fluid pump, connected to an opening of the inner balloon. The PRP pump of the system may inject PRP from the PRP source into the space between the outer balloon and the inner balloon, and then the fluid pump may inflate the inner balloon to force the PRP inside the space between the outer balloon and the inner balloon through the plurality of recessed microneedles into the vaginal cavity through the recessed microneedles.

Optionally, the system may further comprise a programmable controller automating the fluid pump and the PRP pump, and a power source delivering power to the programmable controller, the fluid pump, and the PRP pump. The power source may be a battery or DC power. In other embodiments, the outer balloon and/or inner balloon may comprise biocompatible materials. The recessed microneedles of the outer balloon may be one-way valves. The length of the recessed microneedles may also be less than one (1) centimeter.

In another aspect, a method of delivering platelet-rich plasma to a vaginal cavity for vaginal rejuvenation is also provided. In an exemplary embodiment, such a method may comprise inserting a PRP delivery device into the vaginal cavity, injecting PRP from a PRP source into a cavity between an outer balloon of the PRP delivery device and an inner balloon of the PRP delivery device, and inflating the inner balloon to force the PRP inside the cavity through a plurality of recessed microneedles of the outer balloon into the vaginal cavity. Furthermore, in such exemplary embodiments, a method may further provide for a programmable controller controlling the inflating of the inner balloon, and for the outer balloon and/or inner balloon comprising biocompatible materials. Additionally, in such embodiments, the plurality of microneedles may comprise one-way valves, and may have a length less than one (1) centimeter.

### BRIEF DESCRIPTION OF THE DRAWINGS

The novel features believed characteristic of the invention are set forth in the appended claims. The invention itself, however, as well as a preferred mode of use, further objectives and advantages thereof, will be best understood by reference to the following detailed description of illustrative embodiments when read in conjunction with the accompanying drawings, wherein:

FIG. 1 is a perspective view of the PRP delivery system in accordance with an illustrative embodiment.

FIG. 2 is a system view of the PRP delivery system in accordance with a second illustrative embodiment.

FIGS. 3a and 3b are perspective views of the PRP delivery device.

FIG. 4 is a perspective view of the PRP delivery device in accordance with a third illustrative embodiment

### DETAILED DESCRIPTION

In view of the foregoing, through one or more various aspects, embodiments and/or specific features or sub-components, the present disclosure is thus intended to bring out one or more of the advantages that will be evident from the description. The present disclosure makes reference to one or more specific embodiments by way of illustration and example. It is understood, therefore, that the terminology, examples, drawings and embodiments are illustrative and are not intended to limit the scope of the disclosure.

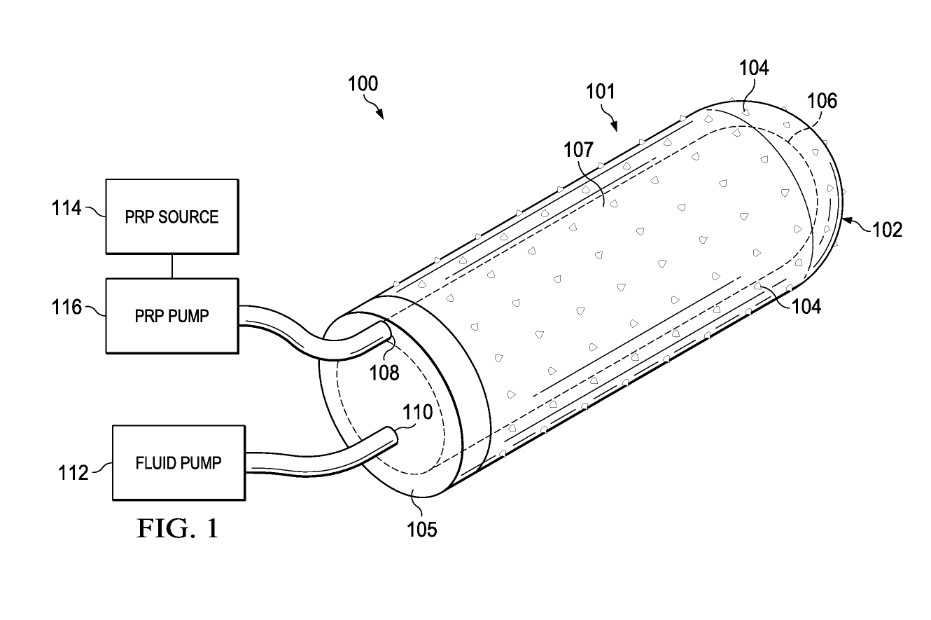

FIG. 1 is a perspective view of the PRP delivery system in accordance with an illustrative embodiment. PRP delivery system 100 can be generally used for delivering PRP into the vaginal cavity for the purpose of vaginal tightening and rejuvenation using a PRP delivery device 101. The PRP delivery device 101, which comprises an outer balloon 102 and an inner balloon 106, is connected to a PRP source 114, a PRP pump 116, and a fluid pump 112. The PRP pump 116 injects PRP from the PRP source 114 through an outer hole 108 of the PRP delivery device 101 to the inter-balloon cavity 105, which is a space between the outer balloon 102 and the inner balloon 106. The fluid pump 112 then inflates the inner balloon 106 by injecting fluid into the inner balloon cavity 107 through an inner hole 110 of the PRP delivery device 101. As fluid inflates the inner balloon 106 of the PRP delivery device 101, the PRP in the inter-balloon cavity 105 is forced toward the outer balloon 102 and through the microneedles 104 recessed in the outer balloon 102 and then into the vaginal cavity.

The PRP delivery device 101 is optimally shaped to match the shape of the vaginal canal and comprises an outer balloon 102 and an inner balloon 106. In one embodiment, the outer balloon 102 is constructed from a semi-rigid material, so that PRP delivery device 101 maintains its shape when the device 101 is inserted into the vaginal cavity. The balloons 102 and 106 can be constructed from polyurethane, polyisoprene, nitrile rubber, or silicone. In one embodiment, the inner balloon 106 is constructed of polyurethane and the outer balloon 102 is constructed of polyisoprene or silicone. In one embodiment, the inner balloon 106 can be constructed from a semi-rigid material, also in order to maintain its shape when the device 101 is inserted into the vaginal cavity and even when there is no PRP inside the inter-balloon cavity 105. The outer balloon 102 can be made of a flexible biocompatible material as the outer balloon 102 is in direct contact with the walls of the vaginal cavity. To facilitate insertion of the PRP delivery device 101 into the vaginal cavity, either the inner balloon 106 or the outer balloon 102 is constructed from a semi-rigid material, and the other balloon is made of a flexible material. A person of ordinary skill in the art would appreciate any combination of biocompatible materials for the outer balloon 102 and the inner balloon 106.

In one embodiment, the outer balloon 102 contacts the walls of the vaginal cavity directly and has recessed microneedles 104 that allow the passage of PRP from the inter-balloon cavity 105 to the vaginal cavity. When the inner balloon 106 is inflated, it compresses against the inside of the outer balloon 102, causes the microneedles 104 to protrude outward toward the walls of the vaginal cavity, and forces the PRP out through the microneedles 104. The microneedles 104 may incorporate a one-way valve for the transmission of the PRP to the vaginal cavity, such as duckbill valves, to prevent contamination due to backflow. The microneedles 104 are recessed in the surface of the outer balloon 102 so that when the PRP delivery device 101 is inserted into the vaginal cavity, the microneedles 104 do not irritate or injure the patient. Additionally, the microneedles 104 do not puncture vaginal tissues to deliver PRP. The thickness of the outer balloon 102 may vary depending on the microneedles 104 because the outer balloon 102 houses and stabilizes the array of microneedles 104. The microneedles 104 may be arranged all over the outer balloon 102 in order to provide maximum PRP transmission coverage.

As mentioned, the PRP pump 116 interacts with the PRP source 114 to inject the PRP through the outer hole 108 into the inter-balloon cavity 105. The PRP is injected into the inter-balloon cavity 105 using any currently existing or later developed pumping mechanism.

The fluid pump 112 interacts with the PRP delivery device 101 to inject fluid through the inner hole 110 into the inner balloon cavity 107. The fluid pump 112 may manually or automatically pump a fluid into the inner balloon cavity 107, and the fluid injected into the inner balloon cavity 107 may be air, liquid, or another shapeless substance. The fluid pump 112 can use any currently existing or later developed pumping mechanism.

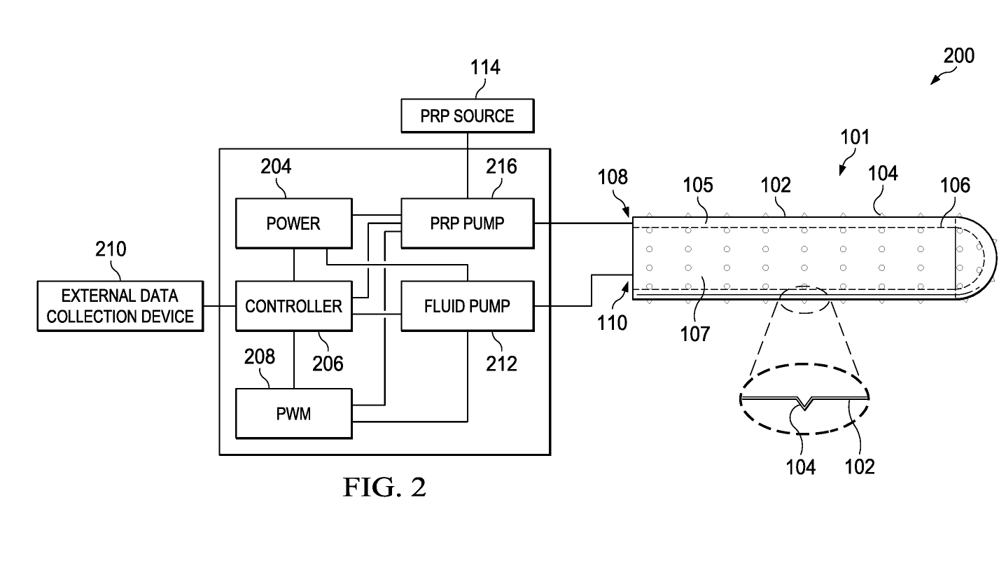

FIG. 2 is a system view of the PRP delivery system 200 in accordance with a second illustrative embodiment. PRP delivery system 200, as mentioned previously, delivers PRP to a vaginal cavity via the PRP delivery device 101. In this second illustrative embodiment, the PRP delivery system 200 is automated and controlled by a programmable controller 206 connected to the PRP pump 216 and fluid pump 212, and to a power source 204.

The programmable controller 206 automates the PRP pump 216 and the fluid pump 212, and controls the injection of PRP into the inter-balloon cavity 105 and the inflation of the inner balloon cavity 107. The programmable controller 206 also monitors the power delivered to the system 200 by the power source 204. The programmable controller 206 may be connected to a pulse width modulator 208 or a programmable digital control to ensure consistent motor speed of the PRP pump 216 or of the fluid pump 212. The programmable controller 206 may incorporate any number of analog to digital converters and digital to analog converters for controlling either the power supply 204 or the motors of the PRP pump 216 and the fluid pump 212. The controller 206 may also be connected to various input/output connectors for connecting to external data collection devices 210. The controller 206 may also comprise a processor and memory devices (or any other tangible non-transitory computer useable medium) for automating the PRP delivery system 200. However, there are any number of ways to implement the control functions described in these disclosures as is known by those with skill in the art.

The programmable controller 206 may be connected to a computer, smartphone, tablet, or any other computing device. By connecting the programmable controller 206 to a computing device, the PRP delivery system 200 may be programmed to perform certain tasks, like those further disclosed below. A computing device may be any electrical device capable of accepting stored program instructions from a computer readable medium and processing those program instructions to perform a defined task. Such devices include, but are not limited to, a mainframe, workstation, desktop, laptop, notebook, or tablet computer, a database server, web server, or the like. One of ordinary skill in the art will appreciate that the construction, choice of programming language, programming, operation, and functionality of such computer processing devices is well known, rendering further description of such devices unnecessary in this regard.

The PRP delivery system 200 may be extemporaneously programmed in an ad-hoc fashion based on information provided by a user. A user may program the controller 206 to automate the PRP system 200 in certain ways. For example, the user may program the PRP delivery system 200 to inflate the inner balloon 106 at a lower rate as compared to the default rate, and so the rate of discharge of PRP into the vaginal cavity is also lower compared to the default rate of discharge into the vaginal cavity.

In another embodiment, the inter-balloon cavity 105 may be partitioned and tubes run inside the inter-balloon cavity 105 to the partitions in the inter-balloon cavity 105. The tubes, in turn, may run through the outer hole 108 to the PRP pump 216, and the programmable controller 206 can direct the flow of PRP through the tubes to certain partitions of the inter-balloon cavity 105. By directing the flow of PRP through the tubes to certain partitions of the inter-balloon cavity 105, when the PRP delivery device 101 is in use, PRP is directed to microneedles 104 corresponding to the certain partitions of the inter-balloon cavity 105.

Alternatively, the microneedles 104 may comprise programmable micro valves that may communicate with the programmable controller 206, so that the programmable controller 206 may be able to control the flow of the PRP into the vaginal cavity by transmitting a signal to certain programmable valves to remain closed and another signal to other programmable valves to allow for PRP flow.

The power source 204 supplies power to the controller 206 and to the other components of the PRP delivery system 200. The power supply 204 may be a battery built into the system, or an external power supply, such as an AC/DC adapter. One of ordinary skill in the art would know how to connect the power supply 204 to the controller 206 and optionally to the fluid pump 212 and/or PRP pump 216.

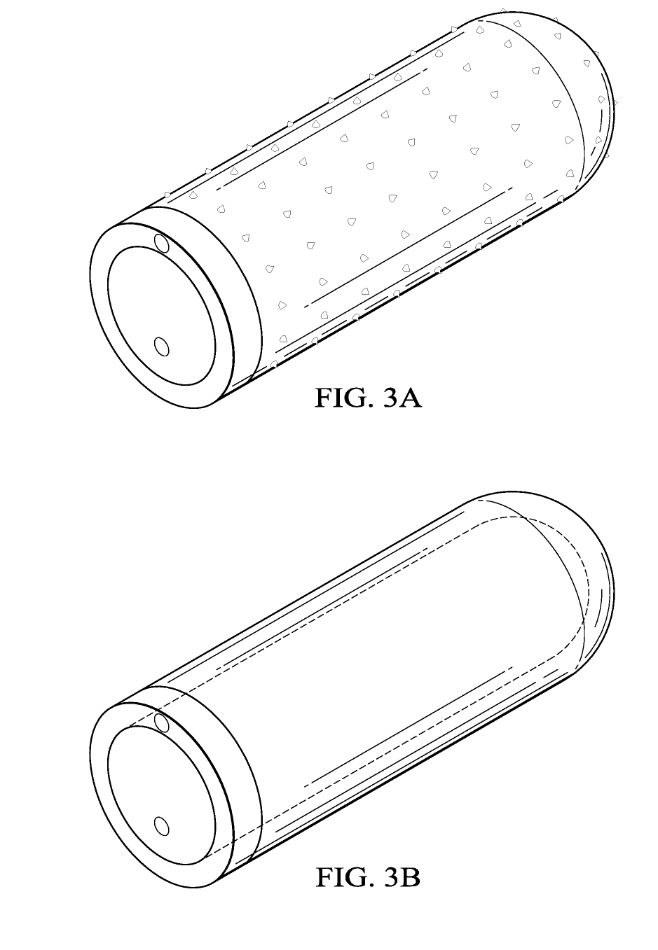

FIGS. 3a and 3b are perspective views of the PRP delivery device that can be used with the system described above with reference to FIG. 2. FIG. 3a is a perspective view of the PRP delivery device that illustrates the arrangement of the microneedles 104 on the outer surface of the outer balloon 102. The microneedles 104, as mentioned previously, may be arranged in any arrangement for the delivery of PRP to the vaginal cavity, and is preferably uniformly arranged so that PRP is uniformly delivered to the vaginal cavity. The microneedles 104 may be formed by molding, overmolding, or through thermal forming so as to connect to the microneedles 104 to the outer balloon 102. The microneedles 104 may also be connected the outer balloon 102 through other currently existing or later developed techniques. FIG. 3b illustrates the internal structure of the PRP delivery device 101 with the inner balloon 106 generally situated at the center of the outer balloon 102.

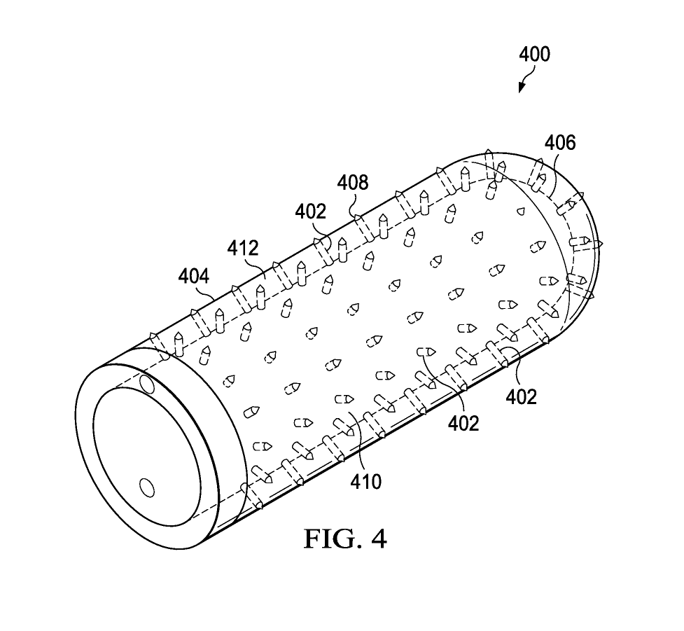

FIG. 4 is a perspective view of the PRP delivery device 400 in accordance with an alternative embodiment. In this alternative embodiment, the roles of the outer balloon 404 and the inner balloon 406 are reversed in that the outer balloon 404 is semi-rigid while the inner balloon 406 is flexible. There are a series of tubes 402 that connect the inner balloon cavity 410 to the microneedles 408. The PRP pump injects the PRP from the PRP source into the inner balloon cavity 410, and the fluid pump injects fluid into the inter-balloon cavity 412. Because, in this illustrative embodiment, the outer balloon 404 is mostly rigid, when the fluid is pumped into the inter-balloon cavity 412, the fluid puts pressure on the outside of the inner balloon 406, causing it to collapse and force the PRP contained inside the inner balloon 406 to move into the tubes 402 and through the microneedles 408. This inward pressure is a reversal of the direction of pressure as compared to the first illustrative embodiment, but the effect is the same in that it forces the PRP to move from inside the PRP delivery device 404 to the vaginal cavity through the microneedles 408.

The invention may be embodied in other specific forms without departing from the spirit or essential characteristics thereof. The present embodiments are therefore to be considered in all respects as illustrative and not restrictive. Accordingly, the scope of the invention is established by the appended claims rather than by the foregoing description. All changes which come within the meaning and range of equivalency of the claims are therefore intended to be embraced therein. Further, the recitation of method steps does not denote a particular sequence for execution of the steps. Such method steps may therefore be performed in a sequence other than recited unless the particular claim expressly states otherwise.

What is claimed is:

1. A system for delivering platelet-rich plasma to a vaginal cavity for vaginal rejuvenation, comprising:
an outer balloon comprising a plurality of recessed microneedles;
an inner balloon inside the outer balloon;
a PRP pump, connected to a space between the outer balloon and the inner balloon and connected to a PRP source;
an fluid pump, connected to an opening of the inner balloon;
wherein
the PRP pump injects PRP from the PRP source into the space between the outer balloon and the inner balloon; and
the fluid pump inflates the inner balloon to force the PRP inside the space between the outer balloon and the inner balloon through the plurality of recessed microneedles into the vaginal cavity.

2. The system of claim 1, further comprising
a programmable controller automating the fluid pump and the PRP pump; and
a power source delivering power to the programmable controller, the fluid pump, and the PRP pump.

3. The system of claim 2, wherein the power source comprises at least one of a battery and DC power.

4. The system of claim 1, wherein the outer balloon comprises biocompatible materials.

5. The system of claim 1, wherein the inner balloon comprises biocompatible materials.

6. The system of claim 1, wherein the plurality of microneedles comprise one-way valves.

7. The system of claim 1, wherein the fluid pump is connected to a fluid source.

8. The system of claim 1, wherein the length of plurality of recessed microneedles is less than 1 cm.

9. A method for delivering platelet-rich plasma to a vaginal cavity for vaginal rejuvenation, comprising:
inserting a PRP delivery device into the vaginal cavity;
injecting PRP from a PRP source into a cavity between an outer balloon of the PRP delivery device and an inner balloon of the PRP delivery device; and
inflating the inner balloon to force the PRP inside the cavity through a plurality of recessed microneedles of the outer balloon into the vaginal cavity.

10. The method of claim 9, wherein a programmable controller controls the inflating of the inner balloon.

11. The method of claim 9, wherein the outer balloon comprises biocompatible materials.

12. The method of claim 9, wherein the inner balloon comprises biocompatible materials.

13. The method of claim 9, wherein the plurality of microneedles comprise one-way valves.

14. The method of claim 9, wherein the fluid pump is connected to a fluid source.

15. The method of claim 9, wherein the length of plurality of recessed microneedles is less than 1 cm.
